(19) United States
(12) Patent Application Publication
(10) Pub. No.: US 2010/0305053 A1
(43) Pub. Date: Dec. 2, 2010

(54) USE OF HESPERIDIN OR OF A DERIVATIVE THEREOF FOR THE PREVENTION AND/OR TREATMENT OF SLACKENED SKIN

(75) Inventors: Audrey Gueniche, Rueil Malmaison (FR); Isabelle Castiel, Nice (FR)

Correspondence Address: OLIFF & BERRIDGE, PLC, P.O. BOX 320850, ALEXANDRIA, VA, 22320-4850, US

(73) Assignees: L'OREAL, PARIS (FR); NESTEC SA, VEVEY (CH)

(21) Appl. No.: 12/671,591

(22) PCT Filed: Aug. 1, 2008
(86) PCT No.: PCT/FR08/51453

§ 371 Date: Apr. 15, 2010

(30) Foreign Application Priority Data
Aug. 2, 2007 (FR) 07 56898

(51) Int. Cl. A61K 8/49; A61Q 19/00

(52) U.S. Cl. 514/27

(57) ABSTRACT

The present invention relates to the oral use of an effective amount of hesperidin or of a derivative thereof as a skin finning agent.

The present invention relates to the prevention and/or treatment of skin defined as slackened skin.

Human skin is made up of three compartments namely a superficial compartment, the epidermis, the dermis and a deep compartment, the hypodermis.

The dermis provides the epidermis with a solid support. It is also its feeder element. It is mainly constituted of fibroblasts and of an extracellular matrix, itself composed mainly of collagen, of elastin and of a substance termed ground substance, these components being synthesized by the fibroblasts.

The extracellular matrix (ECM) of human skin is constituted of various molecules responsible for the mechanical strength of the skin, its flexibility, its tonicity and its elasticity, and also the physiologically important functions (hydration, thermal regulation and regulation of the permeability of the skin).

The macromolecules of the ECM have been arbitrarily classified in four families. The first two are constituted of fibrous and structural macromolecules, the collagens and elastin, while the other two are glycoconjugutes (glycoproteins and proteoglycans).

The collagens represent 70% of the proteins of the ECM. Many types of collagen constitute the ECM, including in particular the interstitial collagens (type I, II, III) of fibrillar structure, which are produced essentially by the fibroblasts and are responsible for the cohesion, rigidity and mechanical strength, the basal laminar collagens (type IV) which are synthesized by the adjacent cells and in the skin by the keratinocytes and which play in particular a mechanical role, the collagens which form basal membrane anchoring fibrils (dermis-epidermis link) which are expressed by the epidermal keratinocytes (type VII) or else the nonfibrillar collagens types V and XII, which are not found in the skin.

In particular, collagen type IV is specific for the basal membrane and collagen type VI and collagen type VII (microfibrillar collagens) are found in the dermoepidermal junction.

The firmness and the pressure-resistance of the skin, and in particular its tonicity, depend especially on the collagen fibers of the basal membrane and of the dermoepidermal junction.

More specifically, collagen fibers are constituted of fibrils that are sealed together, thus forming more than ten types of different structures. The solidity of the dermis is in large part due to the entanglement of the collagen fibers, which are packed tightly together in all directions. The collagen fibers thus contribute to the elasticity and to the tonicity of the skin and/or of mucous membranes.

Over time, during weight changes (such as slimming diets, pregnancies, etc.) or hormonal changes (such as during the menopause), the collagen fibers gradually lose their density, their tension and their regular alignment.

Naturally, the collagen fibers are constantly renewed, but this renewal decreases with age, thereby resulting in a thinning of the dermis. This thinning of the dermis may also be due to pathological causes, such as, for example, hypersecretion of corticoid hormones, certain pathological conditions or else vitamin deficiencies.

Similarly, it is also accepted that extrinsic factors such as ultraviolet rays, tobacco or certain treatments (glucocorticoids, vitamin D and derivatives, for example) also have an effect on the skin and on its level of collagen.

Moreover, collagen fibers are sensitive to certain enzymes known as collagenases.

Collagenases belong to a family of enzymes known as metalloproteinases which are themselves members of a family of proteolytic enzymes (endoproteases or endopeptidases). Their overexpression in humans and their activation is linked to many processes, which are sometimes pathological, involving destruction and remodeling of the matrix.

Prolonged exposure to ultraviolet radiation, in particular to type A and B radiation, thus has the effect of stimulating the expression of collagenases, particularly of MMP1, constituting one of the components of photoinduced skin aging.

Moreover, at the menopause, the main modifications relating to the dermis are a decrease in the level of collagen and in the dermal thickness. This results, in menopausal women, in a slackening of the skin and/or of the mucous membranes owing to the fact that the skin has a reduced elasticity.

Finally, in overweight individuals, and more particularly during weight gain, the adipocytes have a tendency to rapidly increase in volume (owing to storage of increasing amounts of lipids). The strong pressure exerted by the adipocytes on the dermis rapidly causes a deformation of the surface of the skin.

In the end, the fibers degenerate and the skin loses its fundamental structures, which results in an impairment of its viscoelastic or biomechanical properties (loss of firmness, of tonicity, of elasticity).

From the biological point of view, in an individual not undergoing any substantial variation in weight, when the fibroblasts are subjected to a normal tissue tension, they actively synthesize collagen, elastin and glycosaminoglycans, which are essential molecules that contribute to reinforcing the supporting tissues of the skin. Similarly, adipocytes overloaded with lipids also exert a tension on the dermis, leading to an overproduction of collagen until fibrosis occurs. This is reflected, in clinical terms, by skin which is more consistent and taut.

On the other hand, during weight loss, and in particular during slimming diets, the rapid destorage of the adipocytes leads to a considerable decrease in the tension exerted by the hypodermis on the supporting tissues. Consequently, since the dermis is no longer under tension, the connected tissue gradually loses its cohesion: loss of attachment of the fibroblasts to the collagen, decrease in the amount of neocollagen, distension of elastin fibers, depolymerization of proteoglycans.

Consequently, the fibroblasts have less interaction with the fibers of the extracellular matrix.

Consequently, it emerges from the above that numerous factors thus lead to the degradation of collagen fibers, with all the consequences that can be envisioned on the structure, the tonicity and/or the firmness of the skin and/of the mucous membranes.

It is therefore possible to understand the importance of collagen and glycosaminoglycans in the structure of tissues, in particular of the skin and/or of the mucous membranes, and the importance of combating degradation thereof in order to thus combat aging, whether it is chronological or photoinduced aging, and the consequences thereof, in particular on thinning of the dermis and/or degradation of collagen fibers, the latter consequence leading in particular to the appearance of flabby skin, the object of the present invention being precisely to combat this.

The use of flavonoids, including hesperidin, for increasing cell proliferation and treating in particular scars, is already known from WO 03/057210.

WO 2005/058255 describes, moreover, the action of particular flavanones, including hesperidin, for treating skin and hair disorders, in particular via the cytoprotectives and anti-inflammatory properties of said flavanones.

Document EP 0 774 249 takes advantage of a synergistic effect observed for a combination of specific flavanones, on the one hand, or for a combination of specific flavanones with a particular ceramide, on the other hand, on keratinocyte differentiation.

Finally, document WO 01/64177 describes the use (by topical administration) of natural isoflavones extracted from soybean, i.e. in particular of genistin, daidzin, daidzein, genistein, glycitein and acetyl and malonyl forms thereof, for the cosmetic treatment of cellulite or for firmness of the skin.

For their part, the inventors have noted that hesperidin and derivatives thereof are found to have a beneficial effect on the tonicity of the skin. Administration thereof firms the skin, and effectively prevents slackening thereof.

The present invention thus relates to the cosmetic use of an effective amount of hesperidin or of a derivative thereof, as a skin firming agent.

The present invention also relates to the cosmetic use of an effective amount of hesperidin or of a derivative thereof, as an agent for preventing and/or treating slackened skin.

Against all expectations, the inventors have in fact noted that hesperidin and derivatives thereof considered according to the invention advantageously show a beneficial skin-restructuring activity, thus improving the tonicity, firmness and smoothing-out of the skin.

Thus, the use of an effective amount of hesperidin or of a derivative thereof makes it possible in particular to treat stretch marks or to prevent the appearance thereof.

Stretch marks show up as scars in the form of slightly hollowed striations having a pinkish or pearlescent coloring. They are mainly located in places where the skin is stretched: stomach, hips, thighs, buttocks, breasts.

A subject of the invention is also the cosmetic use of an effective amount of hesperidin or of a derivative thereof, as an agent for maintaining and/or restoring the tonicity and/or the firmness of the skin.

The invention also relates to the cosmetic use of an effective amount of hesperidin or of a derivative thereof, as an agent for reducing the cellulite appearance and/or the orange peel appearance.

It also relates to the cosmetic use of an effective amount of hesperidin or of a derivative thereof, as an agent for treating stretch marks or preventing the appearance thereof.

According to one particular embodiment, it may involve topical cosmetic use of an amount of hesperidin or of a derivative thereof, as an agent for treating stretch marks or preventing the appearance thereof.

The invention also relates to the cosmetic use of an effective amount of hesperidin or of a derivative thereof, as a restructuring agent and/or an antislackening agent for the skin and/or the mucous membranes.

A subject of the invention is also the use of an effective amount of hesperidin or of a derivative thereof, for the preparation of a composition, especially a cosmetic and/or dermatological composition, in particular for oral administration, intended to prevent and/or treat slackened skin.

A subject of the invention is also the use of an effective amount of hesperidin or of a derivative thereof, for the preparation of a composition, especially a cosmetic and/or dermatological composition, intended to prevent and/or treat skin disorders associated with cutaneous atrophy and/or with a deterioration in collagen synthesis and/or overexpression of matrix metalloproteases.

According to another of its aspects, the present invention relates to the cosmetic use of an effective amount of hesperidin or of a derivative thereof, for preventing and/or treating connective tissue degeneration associated with an abnormality of collagen production, in particular production of collagen type IV and VII.

The invention also relates to a cosmetic process for treating and/or preventing slackened skin and/or stretch marks, comprising the administration, for example orally, to an individual, of an effective amount of hesperidin or of a derivative thereof.

According to one embodiment, said individual may be a pregnant woman. According to another embodiment, said individual may be an individual on a slimming diet.

For the purpose of the present invention, the term “preventing” is intended to mean reducing the risk of occurrence of a phenomenon.

For the purpose of the present invention, the term “effective amount” is intended to mean an amount sufficient to obtain the expected effect.

The hesperidin or a derivative thereof can be formulated in cosmetic or dermatological compositions.

According to one embodiment, the use or the process according to the invention may comprise the topical, oral or parenteral administration of an effective amount of hesperidin or of a derivative thereof.

The term “topical” is intended to mean an administration of the combination according to the invention or of the compositions which comprise it by application to the skin.

According to another embodiment, the use or the process according to the invention may comprise the aerial or subcutaneous administration of an effective amount of hesperidin or of a derivative thereof.

The subcutaneous administration may in particular be carried out by means of a syringe or of a hydropneumatic-compression injection pistol with a high injection frequency.

Topical and oral administrations can be envisioned for the implementation of the invention.

Topical products act however, by definition, locally on the areas to be treated, on which areas they may be unequally distributed, and require careful and repeated applications. They may also, in certain cases, be responsible for cutaneous side reactions, or even discomfort.

In contrast, oral administration has the advantage of acting globally on the entire skin, in its deep layers (dermis, hypodermis), according to a rapid and relatively nonrestrictive method of administration. This is because the metabolites and other active nutrients are in particular distributed within the dermal matrix by means of the bloodstream. Oral administration or administration by patch also have the advantage of a rapid and relatively nonrestrictive method of administration.

According to one preferred embodiment, the cosmetic use according to the invention is therefore carried out orally and the process according to the invention comprises the oral administration of said effective amount of hesperidin or of a derivative thereof.

Hesperidin

Hesperidin belongs to the family of flavanones, which are natural glucoside compounds found mainly in citrus fruit, i.e. fruit of the Citrus genus, for instance oranges, lemons, bitter oranges or alternatively grapes.

They are present predominantly in the peel of citrus fruit, but are also found in large amounts in the pulp, and therefore in the juice of citrus fruit.

Hesperidin is a glucosylated compound comprising a flavanone nucleus of hesperitin (3′,5′,5-trihydroxy-4′-methoxyflavanone) to which is covalently bonded a glucosidic part of rutinose (L-rhamnosyl-(α1→6)-glucose) attached to the hydroxyl group present on the carbon at position 7 of hesperitin.

The term “hesperidin” is thus intended to mean the compound (S)-7-[[6-O-(6-deoxy-α-L-mannopyranosyl)-β-D-glucopyranosyl]oxy]-2,3-dihydro-5-hydroxy-2-(3-hydroxy-4-methoxyphenyl)-4H-1-benzopyran-4-one.

The hesperidin derivatives may be chosen from its aglycone forms, its chalcone forms, its glycolysated forms and its methylated foul's, and also its sulfated or glucuronidated forms which are found as metabolic products in the bloodstream.

The hesperidin derivatives may be obtained by various processes known to those skilled in the art, such as, for example, enzyme treatments, or alternatively may be obtained by synthesis. Glucose-7-hesperitin may thus be prepared by treatment with rhamnosidase or hesperidinase.

As a hesperidin derivative, mention may in particular be made of the following compounds:
the hesperetin compound, composed of the nonglycosylated flavanone nucleus of hesperidin, which has the following formula: (S)-2,3-dihydro-5,7-dihydroxy-2-(3-hydroxy-4-methoxyphenyl)-4H-1-benzopyran-4-one; 3′,5,7-trihydroxy-4′-methoxyflavanone;
α-glucosylhesperidin, which comprises a chain of 1 to 20 glucose residues linked to one another by a 1,4 linkage, the chain of glucose residues itself being linked by a linkage of 1,4-type at position 4 of the glucose residue of hesperidin; these hesperidin derivatives and the process for the preparation thereof are described in particular in patent application EP 0 825 196 and in U.S. Pat. No. 6,048,712;
methylhesperidin compounds, in particular the compound 3¹-methyl-7-(rhamnosyl-2-methylglucosyl)hesperidin and the compound 3¹-methylhesperidin, these compounds, and also the process for the preparation thereof, being described in U.S. Pat. No. 8,587,84;
conjugates of hesperetin and of sulfate or of glucuronide, which are found, with hesperetin, as hesperidin metabolization products in the bloodstream.

According to one embodiment, hesperidin and derivatives thereof chosen from hesperitin and hesperitin glucuronide are preferably used.

In general, the effective amount of hesperidin or of a derivative thereof can be used in a proportion of from 0.00001% to 20% by weight, for example from 0.001% to 10% by weight, relative to the total weight of the composition containing same.

The compositions according to the invention may be in any of the galenic forms normally available for the method of administration selected.

The carrier may be of diverse nature according to the type of composition under consideration.

As regards more particularly the compositions for topical administration, they may be aqueous, aqueous-alcoholic or oily solutions, dispersions of the solution type or dispersions of the lotion or serum type, emulsions of liquid or semi-liquid consistency of the milk type, suspensions or emulsions of the cream type, aqueous or anhydrous gels, microemulsions, microcapsules, microparticles, or vesicular dispersions of ionic and/or nonionic type. The compositions may also be formulated in the form of a patch.

These compositions are prepared according to the usual methods.

In a known manner, the galenical forms dedicated to topical administration may also contain adjuvants that are customary in the cosmetics, pharmaceutical and/or dermatological field, such as hydrophilic or lipophilic gelling agents, hydrophilic or lipophilic active agents, preservatives, antioxidants, solvents, fragrances, fillers, screens, bactericides, odor absorbers and dyestuffs. The amounts of these various adjuvants are those conventionally used in the field under consideration, and for example from 0.01% to 20% of the total weight of the composition. Depending on their nature, these adjuvants may be introduced into the fatty phase and/or into the aqueous phase.

As fats that can be used in the invention, mention may be made of mineral oils, for instance hydrogenated polyisobutene and liquid petroleum jelly, plant oils, for instance a liquid fraction of shea butter, sunflower oil and apricot kernel oil, animal oils, for instance perhydrosqualene, synthetic oils, in particular Purcellin oil, isopropyl myristate and ethylhexyl palmitate, unsaturated fatty acids and fluorooils, for instance perfluoropolyethers. Use may also be made of fatty alcohols and fatty acids, for instance stearic acid, and for example waxes, in particular paraffin wax, carnauba wax and beeswax. Use may also be made of silicone compounds such as silicone oils and, for example, cyclomethicone and dimethicone, and silicone waxes, resins and gums.

As emulsifiers that can be used in the invention, mention may be made, for example, of glyceryl stearate, polysorbate 60, the mixture of cetylstearyl alcohol/oxyethylenated cetylstearyl alcohol comprising 33 mol of ethylene oxide, sold under the name Sinnowax AO® by the company Henkel, the mixture of PEG-6/PEG-32/glycol stearate, sold under the name Tefose® 63 by the company Gattefosse, PPG-3 myristyl ether, silicone emulsifiers such as cetyl dimethicone copolyol and sorbitan mono- or tristearate, PEG-40 stearate, or oxyethylenated sorbitan monostearate (20EO).

As solvents that can be used in the invention, mention may be made of lower alcohols, in particular ethanol and isopropanol, and propylene glycol.

The composition of the invention may also advantageously contain a spring and/or mineral water, in particular chosen from Vittel water, waters from the Vichy basin and La Roche Posay water.

In the case of an oral use in accordance with the invention, the use of an ingestible carrier is preferred.

The ingestible carrier may be of diverse nature according to the type of composition under consideration.

Tablets or lozenges, oral supplements in dry form and oral supplements in liquid form are thus in particular suitable as pharmaceutical or food carriers.

They may, for example, involve food supplements, the formulation of which may be carried out via the usual processes for producing in particular sugar-coated tablets, gel capsules, gels, emulsions, tablets and capsules.

In the case of oral intakes, the daily doses may, for hesperidin or a derivative thereof, range from 0.5 to 2500 mg/d, in particular from 5 to 500 mg/d. The hesperidin or a derivative thereof that can be used according to the invention may, moreover, be formulated with the usual excipients and components for such oral compositions or food supplements, i.e., in particular, fatty and/or aqueous components, humectants, thickeners, preservatives, texturing agents, flavoring agents and/or coating agents, antioxidants, preservatives and dyes that are customary in the food sector.

The formulating agents and excipients for oral compositions, and in particular for food supplements, are known in this field and will not be the subject of a detailed description herein.

Irrespective of the method of administration under consideration, the effective amount of hesperidin or of a derivative thereof may also advantageously be combined with at least one other active agent.

By way of active agents that can be used, mention may be made of vitamin A, B3, B5, B6, B8, C, D, E or PP, curcuminoids, les carotenoids, polyphenol compounds and mineral compounds, sugars, amino acids, sulfur-containing amino acids, 3 and 6 polyunsaturated fatty acids, probiotics, taurine and phytosterols.

In particular, it is possible to use an antioxidant complex comprising vitamins C and E, and at least one carotenoid, especially a carotenoid chosen from β-carotene, lycopene, astaxanthin, zeaxanthin and lutein, flavonoids such as catechins, proanthocyanidins, anthocyanins, ubiquinones, coffee extracts containing polyphenols and/or diterpenes, extracts of chicory, extracts of ginkgo biloba, extracts of grapes rich in proanthocyanidins, extracts of pimento, extracts of soybean, other sources of flavonoids having antioxidant properties, fatty acids, prebiotics, probiotics, taurine, resveratrol, selenium-containing amino acids, and glutathione precursors.

Among the flavonoids, catechins and OPCs (oligomeric proanthocyanidins) are preferably chosen.

Proteins or protein hydrolysates, amino acids, polyols, in particular C₂ to C₁₀ polyols, for instance glycerol, sorbitol, butylene glycol and polyethylene glycol, urea, allantoin, sugars and sugar derivatives, water-soluble vitamins, starch, bacterial extracts or plant extracts, such as those of Aloe Vera, may be more particularly used as hydrophilic active agents in the topological galenical forms.

As regards the lipophilic active agents, retinol (vitamin A) and derivatives thereof, tocopherol (vitamin E) and derivatives thereof, ceramides, essential oils and unsaponifiable materials (tocotrienol, sesamine, gamma-oryzanol, phytosterols, squalenes, waxes and terpenes) may be used.

As active agents that may more particularly be combined with the hesperidin or with a derivative thereof in an oral or topical galenic formula, any of the ingredients commonly used and/or permitted, in particular active agents for use in the prevention and/or treatment of skin complaints, may also be considered.

By way of illustration, mention may be made of vitamins, minerals, essential lipids, trace elements, polyphenols, flavonoids, phytoestrogens, antioxidants such as lipoic acid and coenzyme Q10, carotenoids, probiotics, prebiotics, proteins and amino acids, monosaccharides and polysaccharides, amino sugars, phytosterols and triterpenic alcohols of plant origin.

They are, in particular, vitamins A, C, D, E and PP and group B vitamins. Among the carotenoids, beta-carotene, lycopene, lutein, zeaxanthin and astaxanthin are preferably chosen. The minerals and trace elements particularly used are zinc, calcium, magnesium, copper, iron, iodine, manganese, selenium and chromium (III). Among the polyphenol compounds, polyphenols from grape, from tea, from olive, from cocoa, from coffee, from apple, from blueberry, from elderberry, from strawberry, from cranberry and from onion are also in particular selected. Preferably, among the phytoestrogens, isoflavones in free form or glycosylated form are selected, such as genistein, daidzein, glycitein or else lignans, in particular those from flax and from Schizandra chinensis. The probiotics are preferably chosen from the group constituted of lactobacillae and bifidobacteria. The amino acids or the peptides and proteins containing them, such as taurine, threonine, cysteine, tryptophan or methionine. The lipids preferably belong to the group of oils containing monounsaturated and polyunsaturated fatty acids, such as oleic acid, linoleic acid, alpha-linolenic acid, gamma-linolenic acid, stearidonic acid, long-chain fish omega-3 fatty acids such as EPA and DHA, conjugated fatty acids derived from plants or animals, such as CLAs (Conjugated Linoleic Acids).

Thus, in particular when the hesperidin or a derivative thereof under consideration according to the invention is for oral administration, it can also be combined with at least one nutritional active agent chosen from lycopene, vitamin C, vitamin E and polyphenol compounds.

With a view to oral use, the hesperidin or a derivative thereof may also be combined with other nutritional active agents chosen from:

anti-aging nutritional active agents, such as food antioxidants, nutrients with free-radical-scavenging properties and cofactors of antioxidant endogenous enzymes, vitamins A, C and E, carotenoids, xanthophylls, isoflavones, certain minerals such as zinc, copper, magnesium, selenium, lipoic acid, coenzyme Q10, superoxide dismutase (SOD) or taurine. Among the anti-aging active agents, mention may in particular be made of the unsaponifiable fractions extracted from lipids of plant origin, aloe vera, native or hydrolyzed marine collagen, plant or marine oils rich in omega-3 and omega-6 fatty acids (including gamma-linolenic acid),

photoprotective nutritional active agents such as: antioxidants and free-radical scavengers: vitamins A, C and E, carotenoids, xanthophylls, certain minerals such as zinc, copper, magnesium, selenium, coenzyme Q10, superoxide dismutase (SOD), probiotics,

nutritional ingredients with moisturizing or immunomodulatory properties, such as extract of Polypodium leucotomos, plant or marine oils rich in omega-3 and omega-6 fatty acids, including gamma-linolenic acid, nutritional active agents that are active on the clinical signs of the menopause (for example, hot flashes, etc.), such as isoflavones, lignans, DHEA, extracts of yam, of sage, or of hops, calcium, magnesium, protein hydrolysates, plant or marine oils rich in omega-3 fatty acids,

nutritional ingredients used in the slimming sector, such as extracts of green tea, mate, horse chestnut, cola, caffeine, theobromine, synephrine, bromelain, ephedra, Citrus aurantiurn, calcium, hoodia, garcinia, chitosan, plant fibers (cactus, apples, pineapples, etc.), fennel, blackcurrant, meadowsweet and black radish.

The cosmetic treatment process for preventing and/or treating slackened skin and/or stretch marks of the invention, and in particular the cosmetic treatment processes intended to reduce the cellulite appearance and the orange peel appearance, can be carried out in particular by applying the cosmetic and/or dermatological compositions or combinations as defined above, according to the customary technique for using these compositions.

In the context of a topical administration, it may be advantageous to also perform at least a massaging of the stretched skin areas, in particular the areas where the stretch marks are located, as described above.

According to one embodiment, the cosmetic treatment process according to the invention may be preceded by a slimming diet.

The cosmetic process according to the invention may be carried out by topical or oral, preferably oral, administration, for example daily, of cosmetic and/or dermatological compositions or of the combination according to the invention which may, for example, be formulated in the form of gels, lotions, emulsions or ingestible carriers.

The process according to the invention may comprise a single administration.

According to another embodiment, the administration is repeated, for example, two to three times daily for one day or more, and generally for a sustained period of at least four weeks, or even 4 to 15 weeks, with, where appropriate, one or more periods of interruption.

In the description and in the examples that follow, unless otherwise indicated, the percentages are percentages by weight and the ranges of values written in the form “between . . . and . . . ” include the upper and lower limits specified. The ingredients are mixed, before being formulated, in the order and under conditions that can be readily determined by those skilled in the art.

The examples and figures hereinafter are given by way of nonlimiting illustration of the field of the invention.

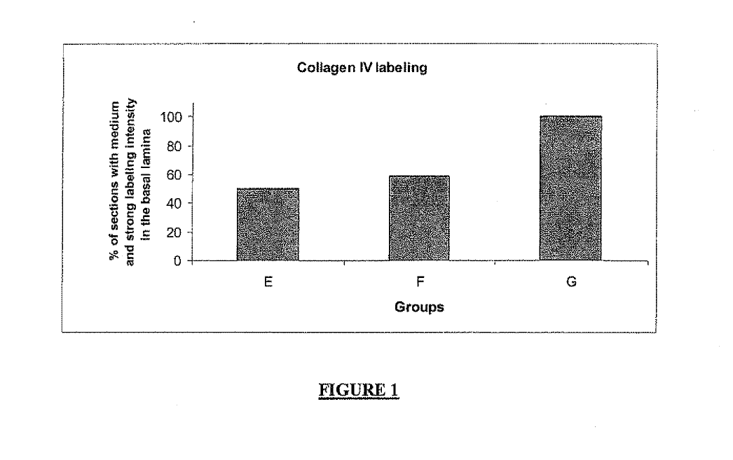

FIG. 1: It reports the expression of collagen IV observed in the dermoepidermal junction in a population of individuals taking an oral hesperidin supplement, relative to a control population.

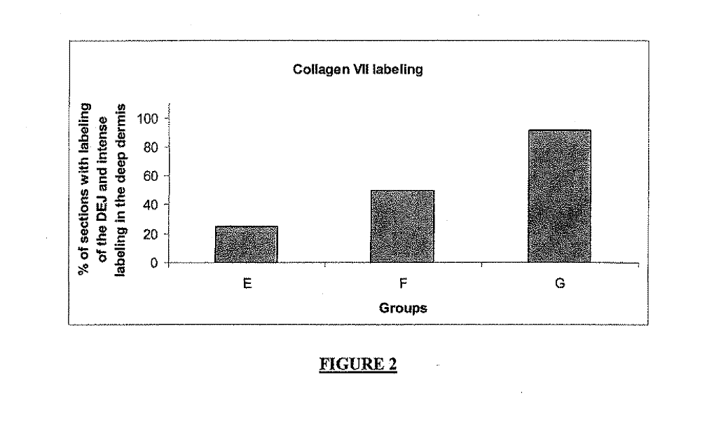

FIG. 2: It reports the expression of collagen VII in the papillary dermis in a population of individuals taking an oral hesperidin supplement, relative to a control population.

### EXAMPLE 1

### Capsule

| | mg/capsule |
|---|---|
| Vitamin C | 60 |
| Hesperidin sold by the company Selectchemie (hesperidin | 15 |
| in micronized form, 93% pure) | |
| Glycerol | 150 |
| Magnesium stearate | 0.02 |
| Natural flavoring | qs |

One to three of these capsules can be taken per day.

### EXAMPLE 2

A vitamin complex comprising 60 mg of vitamin C, 100 μg of vitamin E and 6 mg of β-carotene is added to the formulation of example 2.

### EXAMPLE 3

A vitamin complex comprising 100 mg of vitamin C, 100 μg of vitamin E and 6 mg of lycopene per capsule is added to the formulation of example 2.

### EXAMPLE 4

### Body Lotion in Spray Foam

| | (% by weight) |
|---|---|
| Hesperitin | 5.00 |
| Antioxidant | 0.05 |
| Isopropanol | 40.00 |
| Preservative | 0.35 |
| Water | qs 100.00 |

### EXAMPLE 5

### Firming Bodycare Cream

| | (% by weight) |
|---|---|
| Hesperitin glucuronide | 5.00 |
| Antioxidant | 0.05 |
| Isopropanol | 40.00 |
| Glyceryl stearate | 1.00 |
| Cetylstearyl alcohol/oxyethylenated cetylstearyl | 3.00 |
| alcohol comprising 33 mol EO (Sinnowax AO sold by | |
| the company Henkel) | |
| Cetyl alcohol | 1.00 |
| Dimethicone (DC 200 Fluid sold by the company Dow | 1.00 |
| Corning) | |
| Liquid petroleum jelly | 6.00 |
| Isopropyl myristate (Estol IPM 1514 sold by Unichema) | 3.00 |
| Antioxidant | 0.05 |
| Glycerol | 20.00 |
| Preservative | 0.30 |
| Water | qs 100.00 |

### EXAMPLE 6

### Bodycare Gel

| | (% by weight) |
|---|---|
| Hesperitin | 5.00 |
| Antioxidant | 0.05 |
| Vitamin C | 2.50 |
| Antioxidant | 0.05 |
| Isopropanol | 40.00 |
| Preservative | 0.30 |
| Water | qs 100.00 |

### EXAMPLE 7

### Bodycare Patch (Commercially Available Matricial Transdermal Device)

| | (% by weight) |
|---|---|
| Hesperitin glucuronide | 5.00 |
| Antioxidant | 0.05 |
| Isopropanol | 40.00 |
| Preservative | 0.30 |
| Water | qs 100.00 |

### EXAMPLE 8

### Study of the Effective of Hesperidin on Collagens IV and VII

The study was carried out on elderly, healthy individuals subjected, for 24 weeks, to a diet optionally comprising hesperidin.

The first group of individuals (group E) is a control group subjected to a standard diet free of hesperidin.

The second group of individuals (group F) is a group subjected to a diet comprising 0.1% by weight of hesperidin relative to the standard diet, which corresponds to 50 mg of hesperidin per kg of body weight of the individual and per day.

The third group of individuals (group G) is a group subjected to a standard diet comprising 0.5% by weight of hesperidin relative to the standard diet, which corresponds to 250 mg of hesperidin per kg of body weight of the individual and per day.

Skin samples were taken and analyzed in order to study, by immunofluorescence, the expression of the collagen IV and collagen VII skin markers characteristic of the dermoepidermal junction.

a) Effect of Hesperidin on Collagen IV

Experimental Protocol

The immunolabeling of collagen IV is carried out by immunohistochemistry with an “anticollagen type IV polyclonal” primary antibody [Rockland (sold by Tebu-Bio), dilution 1/50] and an “anti-rabbit FITC” secondary antibody [Santa Cruz, dilution 1/100].

The labeling was carried out on frozen sections 5 μm thick derived from skin samples.

The slides were recovered in PBS for five minutes, incubated with the primary antibody for one hour at ambient temperature in the dark, rinsed in PBS twice for five minutes, then incubated with the secondary antibody for one hour at ambient temperature in the dark, and then again rinsed in PBS twice for five minutes.

The slides were then mounted with Vectashield (Vector) containing DAPI (4,6-diamino-2-phenylindole), and then covered with a coverslip.

Results

The collagen IV labeling is located in the epidermis, exclusively at the dermoepidermal junction (DEJ) and in the dermis, in the internal structures.

A greater expression of collagen IV is observed in the two treated groups, compared with the control, in the basal lamina (FIG. 1).

The table below summarizes the number of sections prepared in each group where the strength of the collagen IV labeling was found to be medium and strong at the DEJ:

| | Number of sections (out of 12) with | |
|---|---|---|
| Groups | medium and strong labeling of the DEJ | % |
| E | 6 | 50 |
| F | 7 | 58 |
| G | 12 | 100 |

Hesperidin makes it possible to obtain a restructuring of the collagen network in the dermal tissue and an increase in the expression of the collagen IV, which is one of the markers specific to the dermoepidermal junction.

b) Effect of Hesperidin on Collagen VII

Experimental Protocol

The collagen VII immunolabeling is carried out as indicated previously, but using “anticollagen type VII polyclonal” [EUROMEDEX (sold by Chemicon), dilution 1/500] as primary antibody and “anti-mouse Ig FITC” [Dake, dilution 1/100] as secondary antibody.

Results

The collagen VII labeling is located at the dermoepidermal junction and is sometimes also present in the papillary dermis.

An increase in fluorescence intensity is noted more particularly for group G, at the DEJ and in the papillary dermis (FIG. 2).

The table below summarizes the number of sections prepared in each group with collagen VII labeling at the DEJ and in the papillary dermis, as a function of the various treatments and the associated percentage:

| | Number of sections (out of 12) with labeling of the | |
|---|---|---|
| Groups | DEJ and labeling in the papillary dermis | % |
| E | 3 | 25 |
| F | 6 | 50 |
| G | 11 | 92 |

Hesperidin makes it possible to obtain a restructuring of the collagen network in the dermal tissue and an increase in the expression of collagen VII, which is one of the markers specific to the dermoepidermal junction.

1. The process of claim 12, wherein said effective amount is a skin finning effective amount.

2. The process of claim 12, wherein said effective amount is an amount effective for reducing a likelihood of and/or treating slackened skin.

3. The process of claim 12, wherein said effective amount is an amount effective for maintaining and/or restoring tonicity and/or firmness of the skin.

4. The process of claim 12, wherein said effective amount is an amount effective for reducing cellulite appearance and orange peel appearance of the skin.

5. The process of claim 12, wherein said effective amount is an amount effective for treating stretch marks or reducing the likelihood of the appearance of stretch marks.

6. (canceled)

7. The process of claim 12, wherein the hesperidin derivatives are selected from the group consisting of aglycone forms, its chalcone forms, glycosylated forms and methylated forms of hesperidin, and sulfated or glucuronidated forms of hesperidin that are found as metabolic products in the bloodstream.

8. The process of claim 7, wherein the derivatives have been obtained by enzyme treatments or by synthesis.

9. The process of claim 12, wherein the hesperidin or derivative thereof is selected from the group consisting of hesperitin and hesperitin glucuronide.

10. The process of claim 12, wherein the effective amount of hesperidin or of a derivative thereof is administered in a proportion of from 0.00001% to 20% by weight relative to the total weight of a composition containing said hesperidin or derivative thereof.

11. The process of claim 12, wherein the effective amount of hesperidin or of a derivative thereof is administered in combination with at least one active agent selected from the group consisting of vitamins A, B3, B5, B6, B8, C, D, and PP, curcuminoids, carotenoids, polyphenol, mineral compounds, sugars, amino acids, sulfur-containing amino acids, 3 and 6 polyunsaturated fatty acids, probiotics, taurine and phytosterols.

12. A cosmetic process, comprising orally administering to an individual hesperidin or a derivative thereof in an amount effective for at least one member selected from the group consisting of: maintaining and/or restoring tonicity and/or firmness of skin; treating and/or reducing a likelihood of slackened skin; reducing cellulite appearance of skin; reducing orange peel appearance of skin; and treating and/or reducing a likelihood of stretch marks on skin.

13. The process of claim 12, wherein said individual is a pregnant woman.

14. The process of claim 12, wherein said individual is an individual on a slimming diet.

15. (canceled)

16. The process of claim 10, wherein said proportion is from 0.001 to 10% by weight.
